(19) United States
(12) Patent Application Publication
(10) Pub. No.: US 2011/0118547 A1
(43) Pub. Date: May 19, 2011

(54) ENDOSCOPE APPARATUS

(75) Inventor: AKIHIKO ERIKAWA, Kanagawa (JP)

(73) Assignee: FUJIFILM Corporation, Kanagawa (JP)

(21) Appl. No.: 12/949,085

(22) Filed: Nov. 18, 2010

(30) Foreign Application Priority Data
Nov. 19, 2009 (JP) 2009-263912

(51) Int. Cl. A61B 1/04

(52) U.S. Cl. 600/108

(57) ABSTRACT

An endoscope apparatus includes first and second light sources, an endoscope leading end portion, an irradiation window, an observation window, a light irradiation unit and an emission angle changing unit. The first light source outputs laser light for diagnosis. The second light source outputs laser light for therapy. The laser light for therapy is different in spectrum from the laser for diagnosis. The irradiation window and the observation window are provided in the endoscope leading end portion. The light irradiation unit emits the laser light for diagnosis and the laser light for therapy to the object through the irradiation window. The emission angle changing unit changes an emission angle at which the laser light for therapy is emitted through the irradiation window to be smaller than an emission angle at which the laser light for diagnosis is emitted. The object to be examined is observed through the observation window.

### CROSS-REFERENCE TO RELATED APPLICATIONS

This application claims the benefit of Japanese Patent Application No. 2009-263912, filed Nov. 19, 2009, the entire contents of which are hereby incorporated by reference, the same as if set forth at length.

### BACKGROUND

1. Technical Field

The invention relates to an endoscope apparatus.

2. Description of the Related Art

Recently, technologies of photodynamic diagnosis (PDD) and photodynamic therapy (PDT) have been developed which emit laser light from a leading end of an insertion portion of an endoscope to diagnose and treat a tumor is developed in an interior wall of the body cavity. In the PDD and PDT, a photosensitive material having a tumor affinity property and being sensitive to specific excitation light is administered into the body in advance. In the PDD, laser light for diagnosis, which is excitation light, is applied to a body tissue surface to observe fluorescent light from a part where a concentration of the photosensitive material is increased because of a lesion part in which the tumor such as cancer exists. In the PDT, laser light for therapy having a specific wavelength is applied to the part where the fluorescence occurs with a relatively strong intensity, thereby destroying the lesion tissue of the lesion part.

JP 2006-130183 A and JP 2006-94907 A have proposed endoscope apparatuses for performing the PDD and PDT, for example. The endoscope apparatuses have such a structure that a PDT probe, which emits laser light for diagnosis through an irradiation window provided at a leading end of an insertion portion of the endoscope apparatus to specify a lesion part and then emits laser light for therapy, is inserted in a forceps opening and protruded from the endoscope leading end and emits the laser light for therapy toward the specified lesion part. Thereby, when the laser light for therapy is emitted toward the specified lesion part, it is difficult to aim the laser light for therapy at the lesion part and to continuously focus the laser light on the lesion part with accuracy because the PDT probe is movable independently of the endoscope leading end.

### SUMMARY

One embodiment of the invention provides an endoscope apparatus which can switch between emission of laser light for diagnosis from an endoscope leading end portion and emission of laser light for therapy from the endoscope leading end portion and accurately aim the laser light for therapy at a target.

According to an aspect of the invention, an endoscope apparatus includes first and second light sources, an endoscope leading end portion, an irradiation window, an observation window, a light irradiation unit and an emission angle changing unit. The first light source outputs laser light for diagnosis for use in photodynamic diagnosis. The second light source outputs laser light for therapy for use in photodynamic therapy. The laser light for therapy is different in spectrum from the laser for diagnosis. The endoscope leading end portion is to be inserted into an object to be examined. The irradiation window is provided in the endoscope leading end portion. The observation window is provided in the endoscope leading end portion. The light irradiation unit emits the laser light for diagnosis and the laser light for therapy to the object to be examined through the irradiation window. The emission angle changing unit changes an emission angle at which the laser light for therapy is emitted through the irradiation window to be smaller than an emission angle at which the laser light for diagnosis is emitted. The object to be examined is observed through the observation window.

With the endoscope apparatus, the laser light for diagnosis and the laser light for therapy can be arbitrarily switched therebetween and emitted from the endoscope leading end portion. Therefore, it is possible to perform the PDD and PDT smoothly and repeatedly. Further, it is possible to accurately aim the laser light for therapy at the object to be examined, so that it is possible to positively perform the PDT in high efficiency.

### BRIEF DESCRIPTION OF THE DRAWINGS

FIG. 1 is a conceptual block diagram of an endoscope apparatus according to an embodiment of the invention.

FIG. 2 is an exemplary appearance view of the endoscope apparatus shown in FIG. 1.

FIG. 3 is a flow chart showing an example of a sequence of PDD and PDT procedures.

FIG. 4 show irradiation with laser light for PDD and laser light for PDT and an example of an observation image.

FIG. 5 is a time chart showing emission timings of the laser light for PDD, white light and the laser light for PDT.

FIG. 6 is a time chart showing emission timings of the laser light for PDD, the white light and the laser light for PDT.

FIG. 7 is a time chart showing emission timings of the laser light for PDD, the white light and the laser light for PDT.

FIG. 8 is a time chart showing emission timings of the laser light for PDD, the white light and the laser light for PDT.

FIG. 9 is a time chart showing emission timings of the laser light for PDD, the white light and the laser light for PDT.

FIG. 10 is a conceptual block diagram of an endoscope apparatus according to another embodiment.

FIG. 11A is an enlarged sectional view of a light emission end of an optical fiber.

FIG. 11B is a plan view of the light emission end of the optical fiber.

FIG. 12 is a schematic block diagram showing a configuration example of a light source device, which has a plurality of laser light sources generating white illumination light, and its surroundings.

FIG. 13 is a schematic block diagram showing a configuration example of a light source device, which emits white illumination light and laser light having a central wavelength of 405 nm from a fluorescent material, and its surroundings.

FIG. 14 is a schematic block diagram showing a configuration example of a light source device and its surroundings in the case where irradiation windows of an endoscope leading end portion are provide at four positions.

### DETAILED DESCRIPTION OF EMBODIMENTS

Hereinafter, illustrative embodiments of the invention will be described in detail with reference to the accompanying drawings.

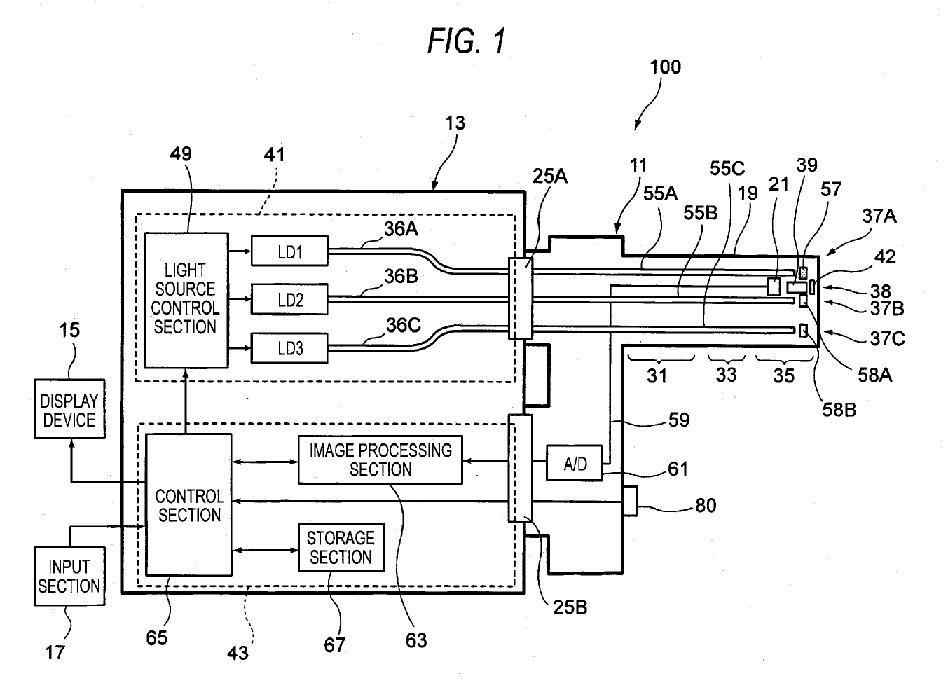

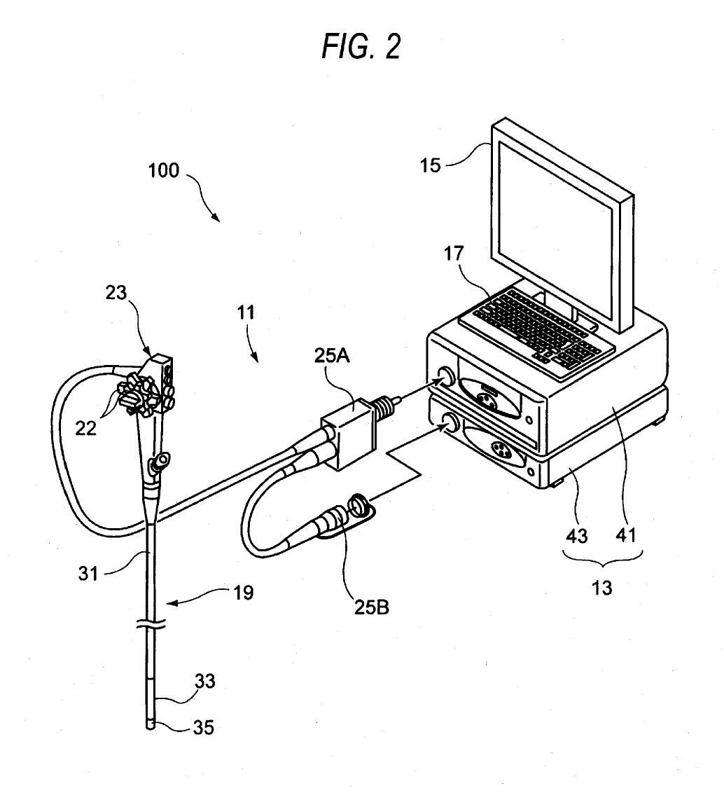

FIG. 1 is a conceptual block diagram of an endoscope apparatus according to one embodiment of the invention. FIG. 2 is an exemplary appearance view of the endoscope apparatus shown in FIG. 1.

As shown in FIGS. 1 and 2, an endoscope apparatus 100 has an endoscope 11 and a control device 13 that is connected to the endoscope 11. The control device 13 is connected to a display device 15 that displays image information and the like and an input section 17 that accepts an input operation. The endoscope 11 is an electronic endoscope having an illumination optical system that emits illumination light from a leading end of an endoscope insertion part 19, which is to be inserted into an object to be examined and an imaging optical system that includes an imaging device 21 (see FIG. 1) imaging an area to be observed.

Also, the endoscope 11 has the endoscope insertion part 19, an operation part 23 (see FIG. 2) with which an operation of bending the leading end of the endoscope insertion part 19 and an observation operation are performed, and connector units 25A, 25B that detachably connect the endoscope 11 to the control device 13. Although not shown, a variety of channels such as forceps channel for inserting a treatment tool for collecting tissue and the like and air supply/water supply channel may be provided in the operation part 23 and the endoscope insertion part 19.

The endoscope insertion part 19 has a flexible part 31 having flexibility, a bending part 33 and a leading end portion (hereinafter, referred to as an “endoscope leading end portion”) 35. As shown in FIG. 1, the endoscope leading end portion 35 is provided with irradiation windows 37A, 37B, 37C through which light is applied to an area to be observed and the imaging device 21, which obtains image information of the area to be observed through an observation window 38, such as a CCD (Charge Coupled Device) image sensor or a CMOS (Complementary Metal-Oxide Semiconductor) image sensor. An optical cut filter 42 which limits specific wavelength components and an object lens unit 39 are disposed between the observation window 38 and the imaging device 21.

The bending part 33 of the endoscope insertion part 19 is provided between the flexible part 31 and the leading end 35 and can be bent by a rotation operation of an angle knob 22 disposed at the operation part 23 shown in FIG. 2. The bending part 33 can be bent in an arbitrary direction at an arbitrary angle depending on parts of an object to be examined for which the endoscope 11 is used, thereby enabling light irradiation directions of the irradiation windows 37A, 37B, 37C of the endoscope leading end portion 35 and a direction in which the imaging device 21 observes through the observation window 38 to be directed toward a desired observation part.

The control device 13 has a light source device 41 that supplies light to the irradiation windows 37A, 37B, 37C of the endoscope leading end portion 35 and a processor 43 that performs image processing for an image signal from the imaging device 21. The control device 13 is connected to the endoscope 11 via the connector units 25A, 25B. In addition, the processor 43 is connected to the display device 15 and the input section 17. The processor 43 performs the image processing for the imaging signal transmitted from the endoscope 11 and generates and supplies an image for display to the display device 15, based on instructions from the operation part 23 of the endoscope 11 and/or the input section 17.

The light source device 41 has a plurality of laser light sources having different central emission wavelengths. In this illustrative embodiment, as shown in FIG. 1, the light source device has a laser light source LD1 having a central emission wavelength of 445 nm, a laser light source LD2 having a central emission wavelength of 405 nm and a laser light source LD3 having a central emission wavelength of 665 nm. The laser light source LD1 is a light source for white illumination that emits blue laser light to generate white illumination light by a wavelength conversion member (which will be described later in detail). The laser light source LD2 is a light source that outputs laser light for diagnosis to perform a photodynamic diagnosis (PDD) (hereinafter, referred to as “laser light for PDD”). The laser light source LD2 is also used as a light source for special-light observation that emits violet laser light. The laser light source LD3 is a light source for performing a photodynamic therapy (PDT) that emits laser light for therapy (hereinafter, referred to as “laser light for PDT”) to a body tissue surface with a relatively strong intensity and treats a tumor such as cancer.

In the PDD, a photosensitive material, which is administered into the body, has a tumor affinity property and is sensitive to a wavelength of the laser light of the laser light source LD2, is excited to emit light in a part where a concentration of the photosensitive material is increased such as a lesion part in which the tumor such as cancer exists. Therefore, a position of a patient's lesion part is specified by detecting a color of the excited luminescence. The laser light for PDT is emitted from the laser light source LD3 to the lesion part specified by the PDD.

A light source control section 49 controls the light from the laser light sources LD1 to LD3 individually. Emission timings and a ratio of amounts of light emitted from the respective laser light sources can be changed arbitrarily.

As the laser light sources LD1 to LD3, InGaN-based laser diodes of a broad area type, InGaNAs-based laser diodes and GaNAs-based laser diodes may be used. In addition, a light emitting body such as light emitting diode may be used as the light sources. With regard to the white illumination light, a xenon lamp or a halogen lamp may be used, in place of the laser light source LD1 and the wavelength conversion member.

The central emission wavelength of the laser light source LD3 may be in a range of 620 to 680 nm. The wavelengths of the laser light sources LD2 and LD3 are appropriately selected depending on a medical agent to be used. For example, as shown in Table 1, when photofrin (and 5-ALA (amino levulinic acid)) is used, the central light emitting wavelength of the laser light source LD2 includes a wavelength component of 405 nm and the central light emitting wavelength of the laser light source LD3 includes a wavelength component of 630 nm. Also, when laserphyrin is used, the central light emitting wavelength of the laser light source LD2 includes a wavelength component of 405 nm and the central light emitting wavelength of the laser light source LD3 includes a wavelength component of 664 nm.

TABLE 1

| | PDD excitation | PDD fluorescent | PDT therapeutic |
|---|---|---|---|
| Medical Agent | light | light | light |
| Photofrin | 405 nm | 630 nm | 630 nm |
| Laserphyrin | 405 nm | 670 nm | 664 nm |
| 5-ALA | 405 nm | 636 nm | 630 nm |

The laser light emitted from the laser light sources LD1 to LD3 are respectively input to optical fibers 36A, 36B, 36C by condensing lenses (not shown) and then transmitted to the connector unit 25A. Optical fibers 55A, 55B, 55C are extended between the connector unit 25A and the endoscope leading end portion 35. The laser light from the laser light source LD1 is introduced into the optical fiber 55A, the laser light from the laser light source LD2 is introduced into the optical fiber 55B, and the laser light from the laser light source LD3 is introduced into the optical fiber 55C, respectively. The laser light from the laser light source LD1 is applied to a fluorescent material 57, which is an example of the wavelength conversion member arranged at the endoscope leading end portion 35, so that white light is emitted through the irradiation window 37A. The laser light from the laser sources LD2 and LD3 are respectively emitted through the irradiation windows 37B, 37C via light deflection/diffusion members 58A, 58B.

The optical fibers 36A, 36B, 36C, 55A, 55B, 55C are multi-mode fibers. As the fibers, a thin fiber cable having a core diameter of 105 μm, a clad diameter of 125 μm and a diameter φ of 0.3 to 0.5 mm, which includes a protective layer serving as an outer sheath, may be used.

The light emitting wavelengths or combination of the respective light sources LD1 to LD3 may be appropriately changed depending on intended use of the endoscope apparatus 100.

The fluorescent material 57 is configured to contain a plurality of fluorescent substances (for example, YAG-based fluorescent substances or BAM (BaMgAl₁₀O₁₇)), which absorb a part of the blue laser light from the laser light source LD1 to emit excited fluorescence of green to yellow. Thereby, the excited fluorescence of green to yellow, which is generated by the blue laser light as the excitation light, and the blue laser light, which passes through the fluorescent material 57 without being absorbed, are combined to generate white (pseudo-white) illumination light.

Here, the white light described in the specification is not strictly limited to light including all wavelength components of the visible light. The white light may be any light so long as it includes light of specific wavelength bands of R (red), G (green) and B (blue) which are reference colors. For example, in a broad sense, the “white light” may include light having wavelength components from green to red and light having wavelength components from blue to green.

Also, the fluorescent material 57 may prevent noise superposition, which is an obstacle to the imaging and flicker that is generated when displaying a moving picture, which are caused due to speckles generated by coherence of the laser light. Considering a difference between a refractive index of the fluorescent substance constituting the fluorescent material and a refractive index of a fixing/solidification resin serving as a filler material, it is preferable that the fluorescent material 57 per se and a diameter of the fluorescent material 57 with respect to the filler material are configured of materials in which light of an infrared region is little absorbed and highly scattered. Thereby, it is possible to increase a scattering effect without decreasing the intensity of light of red or infrared region, so that an optical loss is reduced.

As the light deflection/diffusion members 58A, 58B, a material enabling the laser light from the laser light sources LD2, LD3 to pass through may be used. For example, a light transmitting resin material or glass is used. In addition, the light deflection/diffusion members 58A, 58B may be provided with a light diffusion layer in which particles (filler or the like) having minute unevenness or different refractive indexes are mixed in a surface of a resin material or glass, or may be configured of a semi-transparent material. Thereby, the light transmitted from the light deflection/diffusion member 58 has a uniform amount of light in a predetermined irradiated area.

Also, the light deflection/diffusion members 58A, 58B have different optical characteristics so that the laser light for PDT is applied to a narrower range than the laser light for special-light observation and PDD. In other words, the light deflection/diffusion members 58A, 58B have different lens effects from each other so that an emission angle (a diffusion angle from a light emission axis) of the light emitted from the light deflection/diffusion member 58B is smaller than that of the light emitted from light deflection/diffusion member 58A. Namely, the light deflection/diffusion member 58A serves as an optical lens member which widens an emission angle of light, and the light deflection/diffusion member 58B serves as an optical lens member which narrows an emission angle of light.

As described above, (i) the white light formed of the blue laser light from the laser light source LD1 and the exited luminescent light from the fluorescent material 57 and (ii) the laser light from the laser light sources LD2, LD3 are respectively applied to an area to be observed of an object to be examined through the irradiation windows 37A, 37B, 37C. Switching among the emission of the respective laser light is performed by operating a switch 80 provided in the endoscope 11. An image of the area to be observed to which the illumination light is applied is formed on a light receiving surface of the imaging device 21 by the object lens unit 39 via the light cut filter 42 and the observation window 38, so that a captured image is generated.

The light cut filter 42 has optical characteristics of limiting transmission of wavelength components of the laser light for PDT, which is output from the laser light source LD3 with a relatively high intensity, and allowing light of the visible light range to pass therethrough. The light cut filter 42 may be configured to have an optical characteristic of limiting transmission of the laser light for PDD as well as transmission of the laser light for PDT or limiting transmission of only the laser light for PDD.

An image signal of the captured image output from the imaging device 21 is transmitted to an A/D converter 61 through a scope cable 59 and is then converted into a digital signal. The converted signal is input to an image processing section 63 of the processor 43 through the connector unit 25B. The image processing section 63 performs various processing for the captured image signal, which is output from the imaging device 21 and converted into the digital signal, such as white balance correction, gamma correction, outline emphasis and color correction. The captured image signal, which is processed in the image processing section 63, is transmitted to a control section 65. Then, the control section 65 generates an endoscope observation image based on the captured image signal and various information and displays the endoscope observation image on the display device 15. The endoscope observation image may be stored in a storage section 67 such as a memory or a storage device, if necessary.

Next, a sequence of performing processes of PDD and PDT with the above endoscope apparatus will be described.

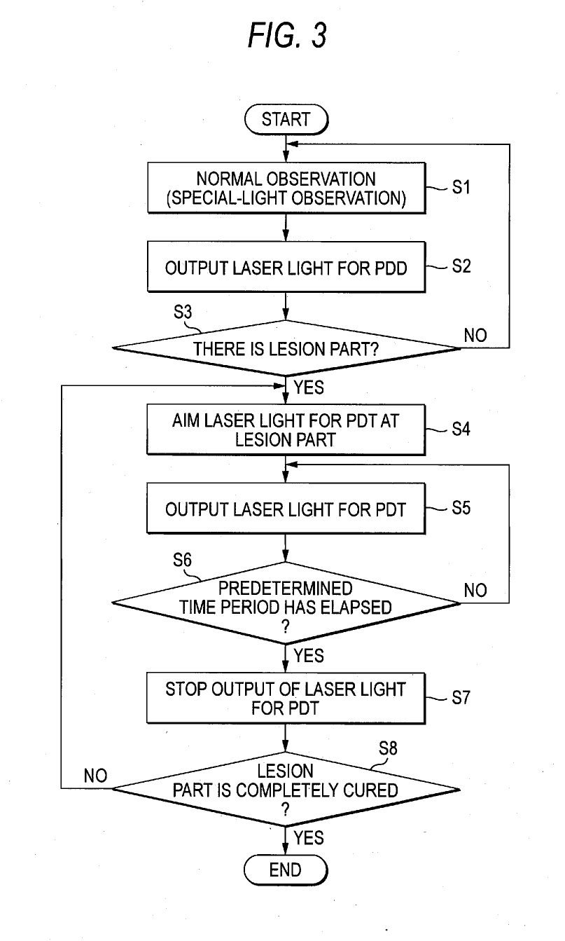

FIG. 3 is a flow chart showing an example of a sequence of PDD and PDT procedures. According to this flow chart, blue laser light is first output from the laser light source LD1 (FIG. 1) to emit white light through the irradiation window 37A having the fluorescent material 57. In addition, purple laser light (narrow-band light), which is laser light for PDD, is output from the laser light source LD2, which is used as illumination light, to perform a special-light observation. As a result, capillary vessels of superficial tissue are emphasized, so that it is possible to easily observe a structure of the blood vessels.

As described above, normal observation under the white light or special-light observation under the illumination light which is obtained by combining the blue or purple laser light with the white light is performed (S1), and the endoscope leading end portion is introduced to an affected area. When the endoscope leading end portion reaches the vicinity of the affected area, outputting of the light from the laser light source LD1 is stopped, and the laser light for PDD is output from the laser light source LD2 (S2). Then, excited luminescence is emitted from a lesion part in which a concentration of the photosensitive material administered to the patient is increased. Therefore, it is checked using fluorescence detection by PDD, as to whether or not there is a lesion part (S3).

The above process is repeated until a lesion part is found. When a lesion part is found, the endoscope leading end portion is approached to the lesion part to aim the laser light for PDT at the lesion part (S4). Specifically, while the laser light for PDD is applied for confirmation so that an area to be irradiated with the laser light for PDD is within a range of φ 20 mm or smaller on an irradiated surface, a position of the endoscope insertion part is adjusted. In the endoscope apparatus 100 having the above configuration, the endoscope leading end portion 35 is provided with the irradiation windows for emitting the laser light for PDD and the laser light for PDT. Therefore, it is possible to simply aim the laser light for PDT by changing the direction of the endoscope leading end portion 35.

After the laser light for PDT is aimed at the lesion part, the laser light source LD3 is driven to emit the laser light for PDT toward the lesion part through the irradiation windows 37A, 37B with relatively high intensity (S5). At this time, light introduced through the observation window 38 is introduced to the imaging device 21 in a limited manner by cutting off components of the laser light for PDT by the light cut filter 42. In irradiation with the laser light for PDT, it is possible to continuously perform the observation with an appropriate exposure without an observation image being saturated, by irradiating with one or both of the laser light for PDD and the white light in addition to the laser light for PDT.

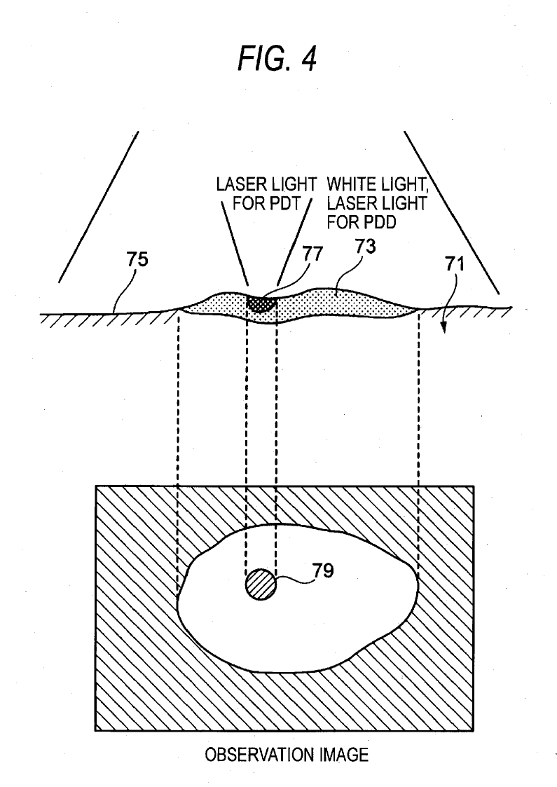

FIG. 4 schematically illustrates irradiation with laser light for PDD and laser light for PDT and an example of an observation image. As shown, when a superficial body tissue, which is an area to be observed in an object 71 to be examined, is irradiated with the laser light for PDD, fluorescence is generated from a lesion part 73, in which a concentration of the photosensitive substance is high, and the fluorescence brightly displays the lesion part 73 an observation image. An area 75 except the lesion part 73 does not emit fluorescence, so that the area 75 is displayed darkly. A position of the lesion part 73 is specified based on the observation image, and the laser light for PDT is emitted toward a specific narrow area (having a diameter of 20 mm or smaller) of the lesion part 73. When the laser light for PDT is irradiated, reflective light of the laser light for PDT is directed to the observation window 38 (see FIG. 1) from an area 77 irradiated with the laser light for PDT. However, the reflective light of the laser light for PDT is cut off by the light cut filter 42, so that the reflective light does not appear on the observation image. The fluorescence generated by the irradiation of the laser light for PDD passes through the light cut filter 42 and thus appears on the observation image.

Accordingly, with the above configuration, when Photofrin or 5-ALA is used as the fluorescent medical agent, it is possible to display the observation image on the display device 15 shown in FIG. 1 by continuously irradiating with the laser light for PDD even during the irradiation with the laser light for PDT. When the treatment of the lesion part by the irradiation with the laser light for PDT is advanced, a concentration of the photosensitive material is decreased in the area 77 irradiated with the laser light for PDT. Thus, it is possible to dynamically observe that an amount of generated fluorescence is decreased. In other words, it is possible to check that as the treatment is advanced, the fluorescence from the area 77 irradiated with the laser light for PDT is gradually darkened in the observation image, and the fluorescence, excited by the laser light for PDD, from the area 77 with irradiated the laser light for PDT is decreased. Namely, it is possible to check on the observation image in real time to what extent the treatment advances.

Accordingly, during the irradiation with the laser light for PDT, it is possible to indirectly check as to whether or not the a position irradiated with the laser light for PDT is deviated, based on an extent to which fluorescence generated by the laser light for PDD is decreased. Thereby, when the irradiated position is deviated from an intended position, for example, it is possible to make adjustment by operating the endoscope 11 so that the lesion part is correctly irradiate with the laser light for PDT, as needed.

Also, without the light cut filter 42 being provided, imaging may be performed by irradiating with the laser light for PDT under the same imaging condition as the case where the white light and the laser light for PDD are irradiated. At this time, halation may occur in an observation image in performing a close zooming observation or the like. However, in this case, if the imaging device is controlled so as to shorten a charge accumulation time of the imaging device during irradiation with the laser light for PDT using an electronic shutter function of the imaging device, it is possible to obtain an observation image with appropriately exposure. Also, appropriate exposure may be obtained by changing between a circuit gain for irradiation with the white light and laser light for PDD and a circuit gain for irradiation with the laser light for PDT. Further, it is possible to simply optimize the imaging conditions by simultaneously using the electronic shutter and changing the circuit gain. Of course, when the laser light for PDT and the laser light for PDD are alternately irradiated, imaging may be performed with making exposure be appropriate for irradiation with each light. Therefore, it is possible to perform proper PDD observation all the time.

It is assumed that the light cut filter 42 is not provided. In this case, for example, even if using Laserphyrin as the fluorescent medical agent causes a wavelength of the PDT treatment light and a wavelength of the PDD fluorescence close to each other, PDD can be performed. That is, since the fluorescence from Laserphyrin is not cut off by the light cut filter 42, the imaging device can detect the fluorescence.

After a predetermined time period has elapsed since the lesion part is irradiated with the laser light for PDT (S6), the irradiation with the laser light for PDT by the laser light source LD3 is stopped (S7), and PDT is ended. Then, it is checked as to whether or not fluorescence is emitted from the position where the lesion part existed by the irradiation with the laser light for PDD. When the fluorescence is emitted from the position of the lesion part, the lesion part is again irradiated with the laser light for PDT, for example. When the fluorescence is not observed, it means that the lesion part is completely cured. Thus, the treatment is ended (S8).

As described above, it is possible to sequentially perform the respective processes of the normal observation or special-light observation, PDD and PDT while the endoscope insertion part is kept being inserted in the patient's body cavity. Further, it is possible to promptly switch between the PDD process and the PDT process by operating the switch 80 or the like. Also, it is possible to correctly aim the laser light for PDT at a desired area by bending or advancing and retreating the bending part 33 of the endoscope insertion part 19, as required. Thereby, the process can be performed efficiently, accurately and promptly, so that it is possible to reduce the burden on the patient.

Here, the special-light observation and the fluorescence observation will be described in detail.

The special-light observation is an observation method of irradiating a body tissue with light having a specific wavelength band which is set in relation to the body tissue and extracting information about the biological tissue, which cannot be obtained through irradiation with the normal white illumination light. For example, by irradiation with narrow-band light having a short wavelength of about 400 nm, it is possible to enhance an image of capillary vessels in superficial mucosa or enhance a fine pattern (pit pattern or the like) of a mucosa surface. This is because hemoglobin in blood in the blood vessels strongly absorbs light having 415 nm among light in the visible wavelength range, and scattering of light by the body tissue gets weak from a short wavelength (blue) to a long wavelength (red). For example, when a position of the blood vessel is irradiated with light having a wavelength of 415 nm, the light is strongly absorbed by hemoglobin, and most of the light is not thus returned to the mucosa surface. To the contrary, the light is little diffused in the surrounding biological tissue of the blood vessel and is reflected and returned as scattering light from the surrounding tissue of the blood vessel. Because of this, an image of the blood vessel is displayed with high contrast. On the other hand, light having a long wavelength (for example, about 500 nm) is less absorbed by hemoglobin as compared with the light of 415 nm, and a part of the light incident on the position of the blood vessel passes through the blood vessel, is scattered by the surrounding tissue and is diffused widely and deeply in the body tissue. Because of this, the narrow-band light having a short wavelength is used for observation of superficial capillary vessels, and the narrow-band light having a long wavelength is used for observation of thick blood vessels.

The fluorescence observation is an observation method of irradiating the body with excitation light and performing diagnosis based on a fluorescence intensity or spectrum of autofluorescence from a fluorescent substance existing in the body tissue or medical agent fluorescence from medical agent administered into the body. The biological tissue includes fluorescent substances such as tyrosine, tryptophan, NADH, FAD, collagen and elastin. In the autofluorescence observation, fluorescent components from the fluorescent substances are observed with being combined. The fluorescence intensity serves as an index of indirectly indicating a change of tumorous lesion as hypertrophy of mucous epithelium or increase of blood flow. Compared to an autofluorescence intensity of a normal mucosa, an autofluorescence intensity from a tumor is remarkably weak. The tumorous tissue has plentiful blood as compared to the normal tissue, and hemoglobin included in the blood strongly absorbs blue light. Thus, the excitation light reaching the fluorescent substances is weakened, so that the autofluorescence is attenuated. In addition, it is said that the tumorous tissue is in a hypoxic state, and the fluorescence intensity is also attenuated by a redox reaction of flavin.

Furthermore, there is a technology of using near infrared light to observe a deep part of a body. When a body is irradiated with light, the light is attenuated due to scattering or absorption. The attenuation of light due to the scattering can be expressed by a function of wavelength. The near infrared light having a long wavelength has a relatively weak scattering property, so that it can reach a deeper part of the body as compared with light having a short wavelength. Since hemoglobin in the blood well absorbs blue light but little absorbs light in a range of from red to near infrared, the near infrared light is suitable for observation of a deep part of the body tissue. Also, as an agent absorbing the near infrared light, there is ICG (indocyanine green) that is used in angiography contrast medium and the like. If ICG is locally administered into a lesion part, it is possible to know a direction of blood flow and a range of blood, which can be used in diagnosis or therapy. As described above, if a body tissue is irradiated with the near infrared light, it is possible to display a thick vein having much hemoglobin therein well. Also, if ICG is administered into a blood vessel, it is possible to observe the blood vessel with better contrast.

Next, emission timings of the laser light from the laser light sources LD1, LD2, LD3 will be described.

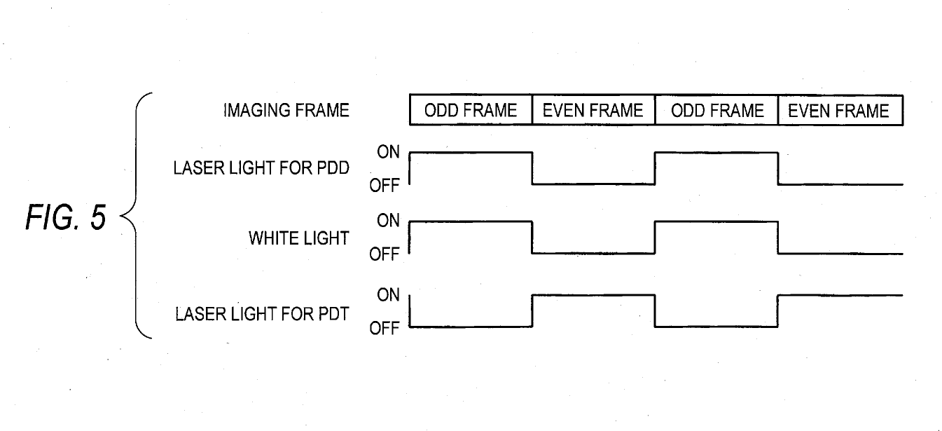

Emission timings of the laser light for PDD, the white light and the laser light for PDT are preferably switched synchronously with imaging frames of the imaging device. FIGS. 5 to 9 show examples of the emission timings. In a pattern shown in FIG. 5, the laser light for PDD and the white light are emitted to perform imaging in odd frames of the imaging frames, and the laser light for PDT is emitted to perform imaging in even frames. In this case, images in which an image of the normal observation and the fluorescent of PDD are superimposed are obtained in the odd frames. Thereby, it is possible to easily confirm an observation location by illumination of the white light, and thus to simply know a position of a lesion part which emits the fluorescence. In the even frame, it is possible to obtain an image showing irradiation with the laser light for PDT. By superimposing and displaying the odd and even frames as one piece of image information, it is possible to simultaneously display images showing execution of PDD and PDT on the observation image of the normal observation. Thereby, it is possible to perform the PDD and PDT more smoothly with high visibility.

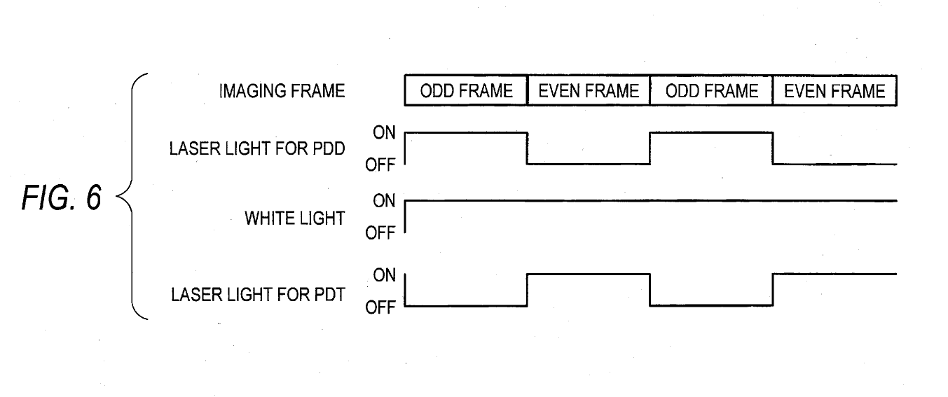

Also, in a pattern shown in FIG. 6, the white light is emitted together with the laser light for PDT in the even frames of the pattern shown in FIG. 5. Thereby, color-reproducibility of the observation images of PDT in the even frames can be improved by the white light. Therefore, it is possible to obtain a more natural image.

It is also possible to display an image of an even frame and an image of an odd frame at different positions in a display area of the display device 15, respectively, without superimposing the image of the even frame and the image of the odd frame as one piece of image information. In this case, it is possible to perform observation or treatment while comparing a lesion part and a treatment part, such as checking the lesion part and the treatment part.

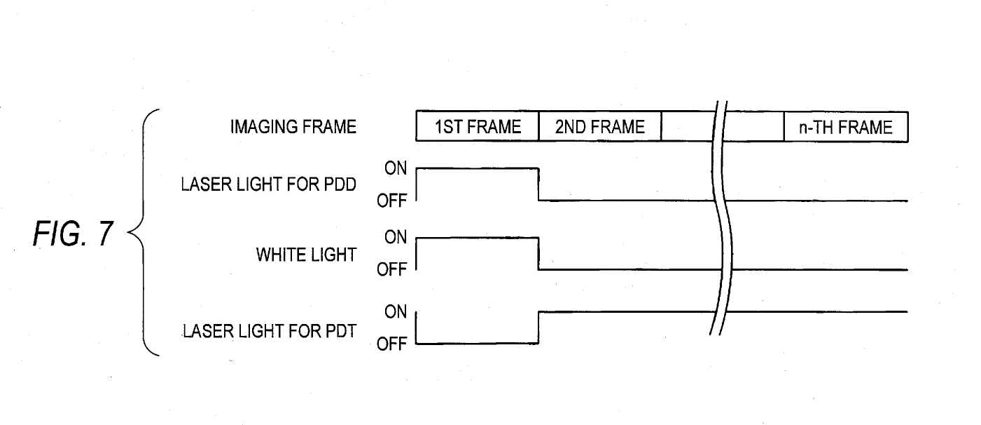

Next, in a pattern shown in FIG. 7, in a first frame, the laser light for PDD and the white light are emitted for observation, and in second to N-th frames, the laser light for PDT is emitted for treatment. With this pattern, it is possible to improve treatment efficiency by continuously irradiating with the laser light for PDT.

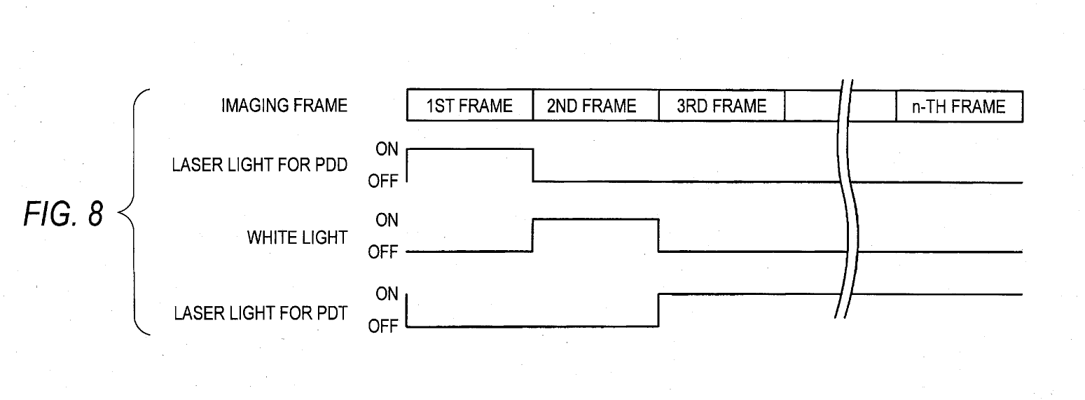

In a pattern shown in FIG. 8, in a first frame, the white light is emitted. In a second frame, the laser light for PDD is emitted for PDD observation. In third to N-th frames, the laser light for PDT is emitted for treatment. With this pattern, the frame in which the white light is irradiated is different from the frame in which the laser light for PDD is irradiated. Therefore, it is possible to easily observe weak fluorescence during PDT.

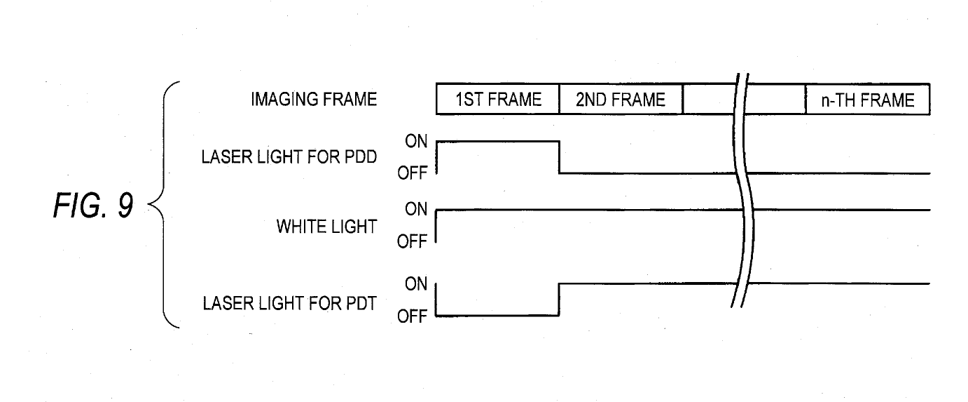

In a pattern shown in FIG. 9, while continuously turning on the white light, in a first frame, the laser light for PDD is emitted together for observation, and in second to N-th frames, the laser light for PDT is emitted while stopping the emission of the laser light for PDD. With this pattern, it is possible to improve treatment efficiency by continuously irradiating with the laser light for PDT.

Next, another configuration example of the endoscope apparatus will be described.

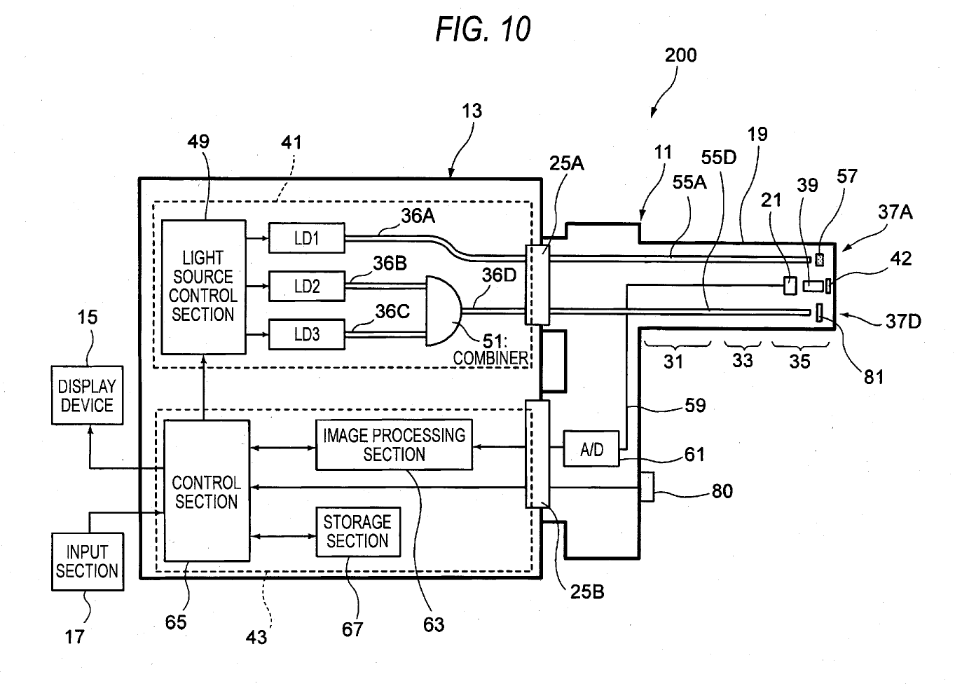

FIG. 10 is a conceptual block diagram of an endoscope apparatus having another configuration. In the following descriptions, constitutional elements common to those shown in FIG. 1 will be indicated by the same reference numerals, and description thereon will be omitted or simplified.

An endoscope apparatus 200 is different from the endoscope apparatus 100 shown in FIG. 1 in that the endoscope apparatus 200 combines light output from the laser light sources LD2, LD3 by a combiner 51 and then transmits the combined light to the endoscope leading end portion 35 and in that a double clad fiber is used to consolidate the optical fibers through which the light output from the laser light sources LD2, LD3 are transmitted. The other structures of the endoscope apparatus 200 are the same as those of the endoscope apparatus 100. Specifically, the endoscope leading end portion 35 has an irradiation window 37A for irradiation of white light and a common irradiation window 37D for laser light for PDD and laser light for PDT. The irradiation window 37D is supplied with light obtained by combining the respective light output from the laser light source LD2 for PDD and laser light source LD3 for PDT. That is, the combiner 51 combines the respective light output from the laser light sources LD2, LD3, and the combined light is transmitted to the connector unit 25A through an optical fiber 36D. Then, in the endoscope 11, the combined output light is transmitted from the connector unit 25A to the irradiation window 37D of the endoscope leading end portion 35 through an optical fiber 55D.

A transparent protection glass 81 is provided at a light emission end of the optical fiber 55D. The laser light for PDD and the laser light for PDT are emitted through the protection glass 81.

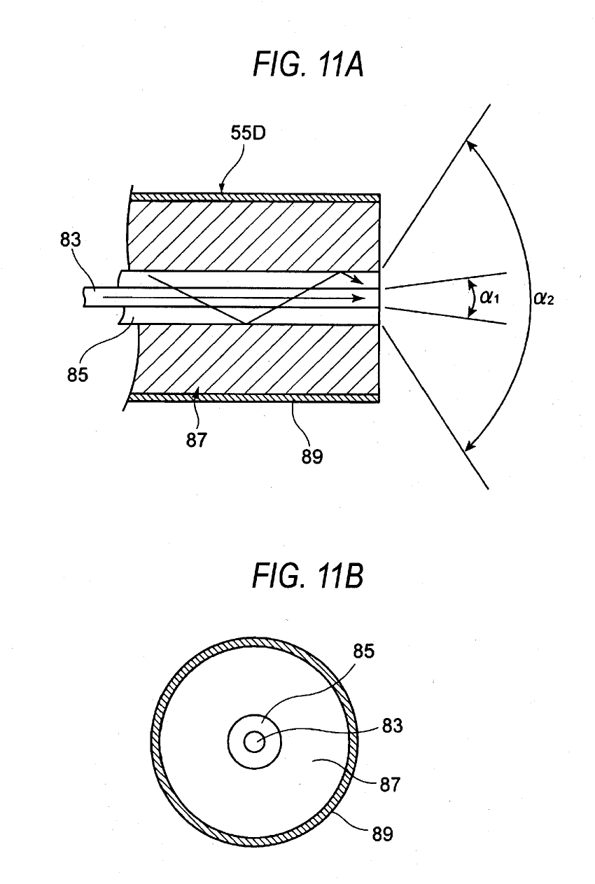

FIG. 11A is an enlarged sectional view of the light emission end of the optical fiber 55D, and FIG. 11B is a plan view of the light emission end. The optical fiber 55D is a double clad fiber including a double-structured clad having a first clad 85 covering an outer side of a core 83 and a second clad 87 covering an outer side of the first clad 85. The first clad and the second clad have different refractive indices. An outer side of the second clad 87 is covered with an outer sheath 89. The refractive indices of the core 83 and the respective clads 85, 87 satisfies a relationship of the refractive index of the second clad 87<the refractive index of the first clad 85<the refractive index of the core 85.

With the configuration of the optical fiber 55D, the laser light for PDT (having a central wavelength of 664 nm) from the laser light source LD3 is transmitted in the core 83 and is emitted at an emission angle α1 from the light emission end. Also, the laser light for PDD (having a central wavelength of 405 nm) from the laser light source LD2 is transmitted in the core 83 and first clad 85 and is emitted at an emission angle α2 from the light emission end. That is, the light transmitted in the optical fiber 55D are classified into the light in the core 83 and the light in the core 83 and first clad 85 and respectively emitted at different emission angles.

Accordingly, from the light emission end of the optical fiber 55D, the laser light for PDD is emitted at the emitting angle α2, and the laser light for PDT is emitted at the emitting angle α1. Then, an object to be examined is irradiated with the emitted light through the protection glass 81.

With the endoscope apparatus 200, it is possible to emit the laser light for PDD and the laser light for PDT through the same light path. Also, it is possible to simply and correctly apply the laser light for PDT to a desired position by aiming the laser light for PDT at a light emission axis of the laser light for PDD. By reversing the above order of the refractive indices of the core 83 and respective clads 85, 87 of the optical fiber 55D, it is also possible to reverse a relation of the emission angles of the respective laser lights.

Next, another configuration example of the light source device 41 will be described.

The laser light sources of the light source device 41 are not limited to the three laser light sources LD1, LD2, LD3. That is, further laser light source(s) may be added.

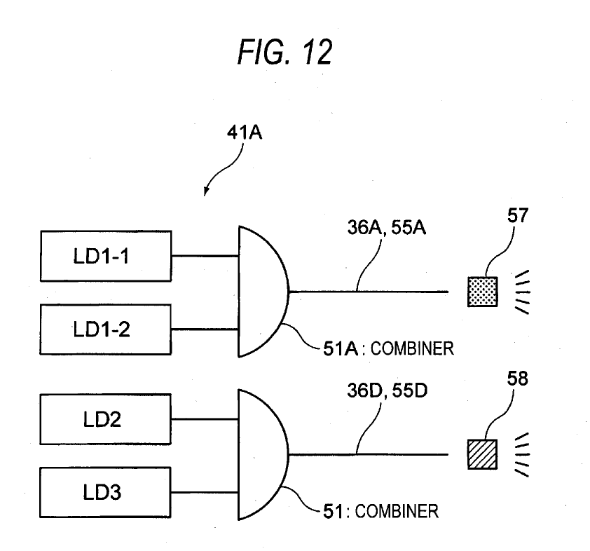

FIG. 12 is a schematic block diagram showing a configuration example of a light source device, which has a plurality of laser light sources generating white illumination light, and its surroundings A light source device 41A has laser light sources LD1-1, LD1-2 that emit laser light having a central wavelength of 445 nm. The laser light output from the respective laser light sources LD1-1, LD1-2 are combined by a combiner 51A and then applied to the fluorescent material 57 of the endoscope leading end portion through the optical fibers 36A, 55A.

With the light source device 41A, the laser light output from the respective laser light sources LD1-1, LD1-2 are combined. Non-uniformity of the wavelengths due to a difference between the individual laser light sources is suppressed. Therefore, it is possible to suppress a change in color of the light emitted from the fluorescent material 57.

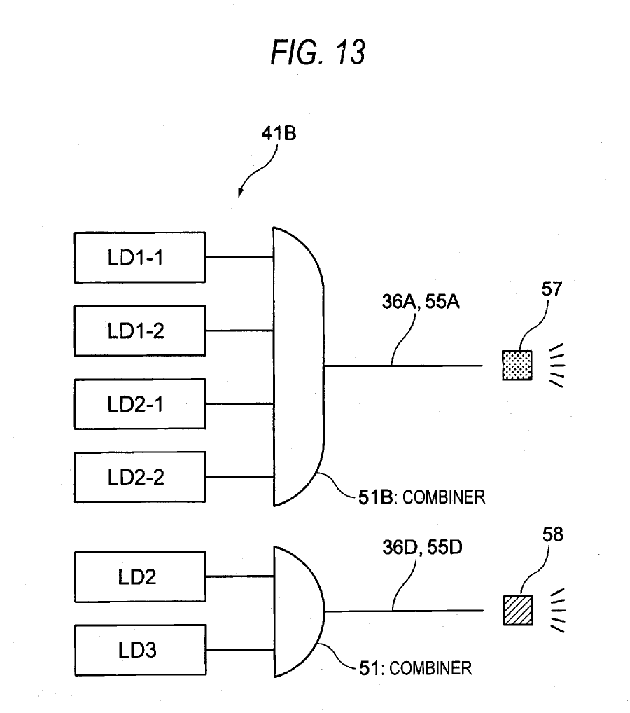

Also, FIG. 13 is a schematic block diagram showing a configuration example of a light source device, which emits white illumination light and laser light having a central wavelength of 405 nm from the fluorescent material 57, and its surroundings. The light source device 41B has laser light sources LD1-1, LD1-2 that generate white illumination light having a center wavelength of 445 nm and laser light sources LD2-1, LD2-2 that generate laser light for special-light observation and PDD having a center wavelength of 405 nm. The laser light output from the respective laser light sources LD1-1, LD1-2, LD2-1, LD2-2 are combined by a combiner 51B and then applied to the fluorescent material 57 of the endoscope leading end portion through the optical fibers 36A, 55A. As the fluorescent material 57, a material having a characteristic of less absorbing wavelength components of the laser light sources LD2-1, LD2-2 is used. Thereby, when the light output from the laser light sources LD2-1, LD2-2 are applied to the fluorescent material 57, the laser light is diffusively emitted while suppressing the excitation of the fluorescent material 57.

With the light source device 41B, it is possible to suppress a change in color of the light emitted from the fluorescent material 57 and to diffusively emit the light output from the laser light sources LD2-1, LD2-2. Also, the plurality of light sources having the same wavelength is provided. Therefore, it is possible to continue or terminate a process procedure in another light source even if one light source is out of order.

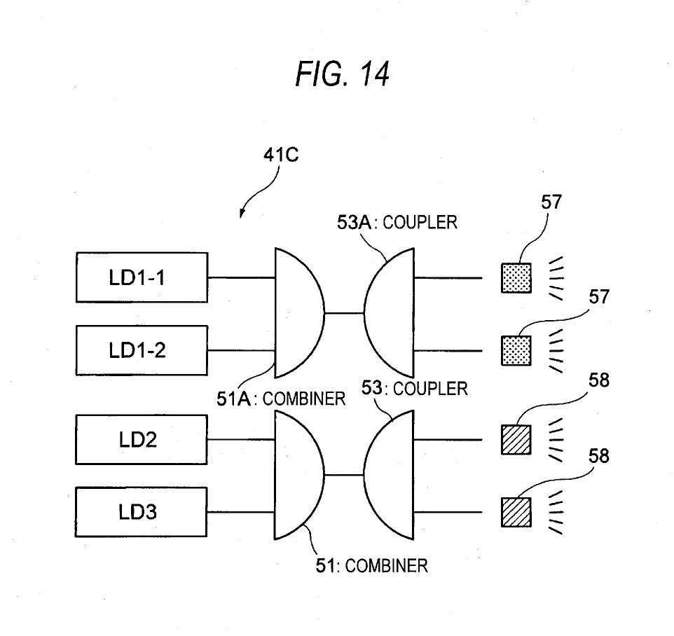

In addition, FIG. 14 is a schematic block diagram showing a configuration example of a light source device and its surroundings in the case where irradiation windows of an endoscope leading end portion are provide at four positions. The light source device 41C has laser light sources LD1-1, LD1-2 that generate white illumination light having a center wavelength of 445 nm, a laser light source LD2 that generates laser light for special-light observation and PDD having a center wavelength of 405 nm, and a laser light source LD3 for PDT. The light output from the laser light sources LD2, LD3 are combined by the combiner 51, divided into a plurality of light paths by a coupler 53 and then emitted through the light deflection/diffusion members 58 disposed at the light emission ends of the respective light paths. Also, the light output from the laser light sources LD1-1, LD1-2 are also combined by the combiner 51A and divided into a plurality of light paths by a coupler 53A. White illumination light is generated by the fluorescent materials 57 disposed at the light emission ends of the respective light paths.

With the light source device 41C and the light deflection/diffusion members 58, the plurality of irradiation windows, through which the light of the same type is emitted, is provided. Therefore, it is possible to apply the light over a broad range to an object to be examined without non-uniformity and to thus prevent a shade from being generated in an observation image.

The invention is not limited to the above illustrative embodiments and can be modified and changed by one skilled in the art based on the descriptions of the specification and the well-known techniques, which are intended to be included in the scope of the invention to be protected.

As described above, the specification describes at least the followings:

(1) An endoscope apparatus includes a first light source, a second light source, an endoscope leading end portion, an irradiation window, an observation window, a light irradiation unit, and an emission angle changing unit. The first light source outputs laser light for diagnosis for use in photodynamic diagnosis. The second light source outputs laser light for therapy for use in photodynamic therapy. The laser light for therapy is different in spectrum from the laser for diagnosis. The endoscope leading end portion is to be inserted into an object to be examined. The irradiation window is provided in the endoscope leading end portion. The observation window is provided in the endoscope leading end portion. The light irradiation unit emits the laser light for diagnosis and the laser light for therapy to the object to be examined through the irradiation window. The emission angle changing unit changes an emission angle at which the laser light for therapy is emitted through the irradiation window to be smaller than an emission angle at which the laser light for diagnosis is emitted. The object to be examined is observed through the observation window.

With the endoscope apparatus, it is possible to arbitrarily switch and emit the laser light for diagnosis and the laser light for therapy from the same endoscope leading end portion. Therefore, it is possible to repeatedly perform the photodynamic diagnosis and photodynamic therapy. Also, since the laser light for diagnosis and the laser light for therapy are emitted through the irradiation window of the endoscope leading end portion, it is possible to adjust the emitting directions of the respective light using the direction of the endoscope leading end portion. Thus, it is possible to easily aim the laser light for therapy. Furthermore, since the laser light for therapy is applied to a specific range which is narrower than an irradiation range of the laser light for diagnosis, it is possible to omit the process of advancing and retreating the endoscope leading end portion in performing the photodynamic diagnosis and photodynamic therapy. Thus, it is possible to surely perform the diagnose and treatment in high efficiency.

(2) In the endoscope apparatus of (1), the light irradiation unit may include a combining unit and an optical fiber. The combining unit combines the laser light for therapy and the laser light for diagnosis. The optical fiber transmits the combined laser light to the endoscope leading end portion.

With the endoscope apparatus, since the laser light for diagnosis and the laser light for therapy are combined and then introduced into the optical fiber, it is possible to unify the light paths through which the laser light are transmitted to the endoscope leading end portion. Thereby, it is possible to make a diameter of the endoscope leading end portion smaller.

(3) In the endoscope apparatus of (2), the optical fiber may include a double-structured clad. The double structured core has a core, a first clad that covers an outer side of the core, and a second clad that covers an outer side of the first clad and is different in refractive index from the first clad. The laser light for therapy is transmitted in the core. The laser light for diagnosis is transmitted in the core and the first clad. The transmitted laser light for therapy and the transmitted light for diagnosis are emitted at the different emission angles from a light emission end of the optical fiber.

With the endoscope apparatus, the laser light are transmitted in the different areas such as in the core and in the core and first clad depending on the wavelengths of the laser light to be transmitted. Therefore, the laser light are emitted at different emission angles when they are emitted from the light emission end of the optical fiber.

(4) In the endoscope apparatus of (1), the emission angling change unit may include an optical lens member that is disposed on at least one of a light path of the laser light for diagnosis and a light path of the laser light for therapy.

With the endoscope apparatus, it is possible to change the emission angles of the laser light for diagnosis and the laser light for therapy through the irradiation windows with a simple configuration, that is, by the optical lens member arranged on the light path.

(5) The endoscope apparatus of any one of (1) to (4) may further include a light source for white illumination that supplies white light to the light irradiation unit.

With the endoscope apparatus, the light for emitting white light through the irradiation window is supplied from the light source for white illumination. Therefore, it is possible to perform the white light illumination (normal illumination).

(6) In the endoscope apparatus of (5), the irradiation window of the endoscope leading end portion may include a first irradiation window through which the laser light for diagnosis and the laser light for therapy are emitted, and a second irradiation window through which the white light is emitted.

With the endoscope apparatus, since the laser light for diagnosis and the laser light for therapy are emitted via the common optical system through the first irradiation window, it is possible to make a diameter of the endoscope leading end portion smaller. Also, since the laser light for therapy is emitted through the same irradiation window as the laser light for diagnosis, it is possible to easily aim the laser light for therapy. Further, since the white light is emitted through the irradiation window different from the laser light for diagnosis and the laser light for therapy, it is possible to improve a degree of freedom of designs regarding the irradiation direction of the illumination light, the arrangement of the irradiation windows and the like.

(7) The endoscope apparatus of (1) may further include a light source for white light illumination and a wavelength conversion member. The light source for white light illumination outputs laser light for white light illumination. The irradiation window of the endoscope leading end portion may include a first irradiation window through which the laser light for diagnosis and the laser light for therapy are emitted, and a second irradiation window through which the white light is emitted. The wavelength conversion member is dispose on an inner side of the second irradiation window. The wavelength conversion member wavelength-converts a part of the laser light for white light illumination output by the light source for white light illumination. The wavelength-converted light and the laser light for white illumination generate white light. The generated white light is supplied to the light irradiation unit.

With the endoscope apparatus, it is possible to generate the white light of high brightness in high efficiency by the laser light for white illumination and the light having a wavelength converted by the wavelength conversion member. Also, since the laser light for diagnosis and the laser light for therapy are emitted through the irradiation window which is different from that for the white light, it is not necessary to provide the wavelength conversion member on the light path(s) of the laser light for diagnosis and the laser light for therapy. Thereby, it is possible to suppress the light loss of the laser light for diagnosis and the laser light for therapy and to prevent the unnecessary light from being generated.

(8) In the endoscope apparatus of (7), the light source for white illumination may include plural light sources for white illumination. The light output by the light source for white illumination are combined and supplied to the light irradiation unit.

With the endoscope apparatus, even if there is an error between the light emitting wavelengths due to a difference between the individual light sources for white illumination, the light from the light sources of white illumination are combined to average the errors. Therefore, a color of the excited fluorescence from the fluorescent material is maintained as prescribed. Thereby, it is possible to make the white illumination light have the prescribed color with high precision.

(9) The endoscope apparatus of any one of (5) to (8) may further include an imaging device and a light source control unit. The imaging device images the object to be examined through the observation window. The light source control unit controls emission timings of the laser light for diagnosis, the laser light for therapy and the white light in synchronous with an imaging timing of the imaging device.

With the endoscope apparatus, the light source control unit performs the light emission in synchronous with the imaging timing of the imaging device. Therefore, it is possible to perform the observation and treatment of the lesion part simultaneously. For example, treat can be advanced while observing, in real time, an extent to which generation of fluorescence by the laser light for diagnosis is decreased as the treatment by irradiation of the laser light for therapy advances.

What is claimed is:

1. An endoscope apparatus comprising:
a first light source that outputs laser light for diagnosis for use in photodynamic diagnosis;
a second light source that outputs laser light for therapy for use in photodynamic therapy, wherein the laser light for therapy is different in spectrum from the laser for diagnosis;
an endoscope leading end portion to be inserted into an object to be examined;
an irradiation window provided in the endoscope leading end portion;
an observation window provided in the endoscope leading end portion;
a light irradiation unit that emits the laser light for diagnosis and the laser light for therapy to the object to be examined through the irradiation window; and
an emission angle changing unit that changes an emission angle at which the laser light for therapy is emitted through the irradiation window to be smaller than an emission angle at which the laser light for diagnosis is emitted, wherein
the object to be examined is observed through the observation window.

2. The endoscope apparatus according to claim 1, wherein
the light irradiation unit includes
a combining unit that combines the laser light for therapy and the laser light for diagnosis, and
an optical fiber that transmits the combined laser light to the endoscope leading end portion.

3. The endoscope apparatus according to claim 2, wherein
the optical fiber includes a double-structured clad having
a core,
a first clad that covers an outer side of the core, and
a second clad that covers an outer side of the first clad and is different in refractive index from the first clad,
the laser light for therapy is transmitted in the core,
the laser light for diagnosis is transmitted in the core and the first clad, and
the transmitted laser light for therapy and the transmitted light for diagnosis are emitted at the different emission angles from a light emission end of the optical fiber.

4. The endoscope apparatus according to claim 1, wherein the emission angling change unit includes an optical lens member that is disposed on at least one of a light path of the laser light for diagnosis and a light path of the laser light for therapy.

5. The endoscope apparatus according to claim 1, further comprising:
a light source for white illumination that supplies white light to the light irradiation unit.

6. The endoscope apparatus according to claim 2, further comprising:
a light source for white illumination that supplies white light to the light irradiation unit.

7. The endoscope apparatus according to claim 3, further comprising:
a light source for white illumination that supplies white light to the light irradiation unit.

8. The endoscope apparatus according to claim 4, further comprising:
a light source for white illumination that supplies white light to the light irradiation unit.

9. The endoscope apparatus according to claim 5, wherein
the irradiation window of the endoscope leading end portion includes
a first irradiation window through which the laser light for diagnosis and the laser light for therapy are emitted, and
a second irradiation window through which the white light is emitted.

10. The endoscope apparatus according to claim 1, further comprising:
a light source for white light illumination that outputs laser light for white light illumination; and
a wavelength conversion member, wherein
the irradiation window of the endoscope leading end portion includes
a first irradiation window through which the laser light for diagnosis and the laser light for therapy are emitted, and
a second irradiation window through which the white light is emitted,
the wavelength conversion member is dispose on an inner side of the second irradiation window,
the wavelength conversion member wavelength-converts a part of the laser light for white light illumination output by the light source for white light illumination,
the wavelength-converted light and the laser light for white illumination generate white light, and
the generated white light is supplied to the light irradiation unit.

11. The endoscope apparatus according to claim 10, wherein
the light source for white illumination includes plural light sources for white illumination,
the light output by the light source for white illumination are combined and supplied to the light irradiation unit.

12. The endoscope apparatus according to claim 5, further comprising:
an imaging device that images the object to be examined through the observation window, and
a light source control unit that controls emission timings of the laser light for diagnosis, the laser light for therapy and the white light in synchronous with an imaging timing of the imaging device.

13. The endoscope apparatus according to claim 6, further comprising:
an imaging device that images the object to be examined through the observation window, and
a light source control unit that controls emission timings of the laser light for diagnosis, the laser light for therapy and the white light in synchronous with an imaging timing of the imaging device.

14. The endoscope apparatus according to claim 7, further comprising:
an imaging device that images the object to be examined through the observation window, and
a light source control unit that controls emission timings of the laser light for diagnosis, the laser light for therapy and the white light in synchronous with an imaging timing of the imaging device.

15. The endoscope apparatus according to claim 8, further comprising:
an imaging device that images the object to be examined through the observation window, and
a light source control unit that controls emission timings of the laser light for diagnosis, the laser light for therapy and the white light in synchronous with an imaging timing of the imaging device.

16. The endoscope apparatus according to claim 9, further comprising:
an imaging device that images the object to be examined through the observation window, and
a light source control unit that controls emission timings of the laser light for diagnosis, the laser light for therapy and the white light in synchronous with an imaging timing of the imaging device.

17. The endoscope apparatus according to claim 10, further comprising:
an imaging device that images the object to be examined through the observation window, and
a light source control unit that controls emission timings of the laser light for diagnosis, the laser light for therapy and the white light in synchronous with an imaging timing of the imaging device.

18. The endoscope apparatus according to claim 11, further comprising:
an imaging device that images the object to be examined through the observation window, and
a light source control unit that controls emission timings of the laser light for diagnosis, the laser light for therapy and the white light in synchronous with an imaging timing of the imaging device.

19. The endoscope apparatus according to claim 12, further comprising:
an imaging device that images the object to be examined through the observation window, and
a light source control unit that controls emission timings of the laser light for diagnosis, the laser light for therapy and the white light in synchronous with an imaging timing of the imaging device.
